(19) United States
(12) Patent Application Publication
(10) Pub. No.: US 2016/0151086 A1
(43) Pub. Date: Jun. 2, 2016

(54) NEEDLE ASSEMBLIES AND METHODS OF MANUFACTURE

(71) Applicant: SMITHS MEDICAL INTERNATIONAL LIMITED, Ashford, kent (GB)

(72) Inventors: Stephen James Field, Kent (GB); Thomas Cuthbert Mills, Kent (GB)

(73) Assignee: SMITHS MEDICAL INTERNATIONAL LIMITED, Ashford, kent TN25 5BF (GB)

(21) Appl. No.: 14/903,073

(22) PCT Filed: Jun. 17, 2014
(86) PCT No.: PCT/GB2014/000237

§ 371 Date: Jan. 6, 2016

(30) Foreign Application Priority Data
Jul. 13, 2013 (GB) 1312600.8

(51) Int. Cl. A61B 17/34; B29C 44/12; B29C 47/02

(52) U.S. Cl. CPC A61B 17/3403; B29C 47/021; B29C 44/12; A61B 2017/3413; A61B 2017/00526

(57) ABSTRACT

A needle assembly of a metal shaft (10, 10′) and an outer plastics sleeve (12) is made by extruding the sleeve onto the outside of the shaft. The sleeve contains gas bubbles (13), preferably with a size in the range 5 μ to 10 μ, to increase the ultrasound visibility of the assembly. A sharp, penetrating tip (18) may be formed on the shaft either before or after the sleeve is extruded on the shaft. The metal shaft (10) may be supplied to the extruder 1 to 5 in a continuous length and cut to the size of the needle assemblies after extrusion of the sleeve (12). Alternatively, pre-cut lengths of metal shafts (10′) could be supplied to the extruder 1′ to 5′, the sleeve being cut between the shafts after extrusion.

This invention relates to needle assemblies of the kind including a hollow needle shaft.

Ultrasound scanners are used increasingly to help direct or check placement of catheters and other devices inserted in the body. Some of these devices are not normally very visible under ultrasound because of their shape, size or the fact that the material from which they are made has similar reflectance acoustic impedance to the tissue or body fluid within which they are inserted. Attempts have been made to increase the visibility of medico-surgical devices under ultrasound observation in various ways. Where the device is of a metal within the body it may reflect ultrasound but the reflected energy tends to be highly directional so it does not necessarily produce a very visible image on the scanner.

The usual way of increasing the visibility of a metal needle is by modifying its surface, such as by forming grooves or indentations in its surface. A reflective liquid coating may be applied to the device, such as incorporating bubbles, as described in WO98/19713. Discrete echogenic markings may be deposited on a device, as described in EP0624342. U.S. Pat. No. 8,398,596 describes a metal needle with a bubble-filled stylet or a removable outer sleeve of a bubble-filled material. Where the device is of a plastics material, such as a catheter of the kind described in GB2379610 the wall may include gas bubbles or a bubble-containing material may be incorporated in a stripe occupying only a part of the circumference. GB2400804 describes a similar catheter with several layers. U.S. Pat. No. 7,258,669 describes a catheter with a helical, gas-filled lumen extending along its length. WO9822022 describes an instrument with an inner stylet that may have an air void or a solution containing microbubbles. DE 102006051978 describes a bubble-filled rod inserted along the bore of a flexible plastics catheter to enhance visibility under ultrasound observation.

The ultrasound visibility of a catheter in a body can also be enhanced by supplying a fluid containing bubbles along the bore of the catheter. These arrangements, however, are not suitable in all cases. It may, for example, be undesirable to paint a substance onto a device because of the risk of detachment. Also, some arrangements do not provide visibility along the length of the device. Altering the surface of a metal device by forming grooves or the like may reduce the smoothness of the device. A sleeve of a bubble-filled material on a needle may be effective but such sleeves can be difficult to manufacture, handle and assemble on a needle where the sleeve has a relatively thin wall.

It is an object of the present invention to provide an alternative needle assembly and method of manufacture.

According to one aspect of the present invention there is provided a needle assembly of the above-specified kind, characterised in that the assembly includes a plastics sleeve extruded onto and extending along the outside of the needle shaft, and that the extruded sleeve contains a plurality of gas bubbles within the thickness of the sleeve such that the sleeve is visible under ultrasound observation and such that the ultrasound visibility of the assembly with the sleeve is greater than that of the needle alone.

The assembly preferably includes a hub joined at one end with the needle shaft and a sharp, penetrating tip at the opposite end. The gas bubbles may have a size range of 0.1 μ to 300 μ and preferably in the range 1 μ to 50 μ, more preferably in the range 5 μ to 10 μ. The sleeve may have a thickness between 0.01 mm and 2 mm and preferably a thickness between 0.01 mm to 0.1 mm.

According to another aspect of the present invention there is provided a method of manufacture of a needle assembly including the steps of providing a metal shaft and extruding on the outside of the shaft a sleeve of a plastics material containing gas bubbles within its thickness, wherein the metal shaft is formed with a sharp, penetrating tip and the gas bubbles are effective to increase ultrasound visibility of the assembly.

The sharp, penetrating tip may be formed before or after the plastics sleeve is extruded on the shaft.

According to a second aspect of the present invention there is provided a method of manufacture of a needle assembly including the steps of providing a metal shaft and extruding on the outside of the shaft a sleeve of a plastics material containing gas bubbles within its thickness, the metal shaft being formed with a sharp, penetrating tip.

The sharp, penetrating tip may be formed before or after the plastics sleeve is extruded on the shaft.

According to a third aspect of the present invention there is provided a method of manufacture of needle assemblies including the steps of supplying a hollow metal shaft to extruder apparatus, extruding around the outside of the shaft in the extruder apparatus a sleeve of a plastics material containing gas bubbles, subsequently cutting the shaft into discrete lengths, and forming a sharp, penetrating tip (18) at one end to form needle assemblies.

According to a fourth aspect of the present invention there is provided a method of manufacture of needle assemblies including the steps of supplying to extruder apparatus a plurality of pre-cut lengths of metal shafts, extruding around the outside of the shafts in the extruder apparatus a sleeve of a plastics material containing gas bubbles, subsequently cutting through the extruded sleeve to form separate shafts, each shaft being formed with a sharp, penetrating tip at one end either before or after supply to the extruder machine.

The method according to the above second, third or fourth aspect of the present invention may include the step of attaching a hub to each shaft at the end opposite the sharp, penetrating tip.

According to a fifth aspect of the present invention there is provided a needle assembly made by a method according to the above other second, third or fourth aspect of the present invention.

A needle assembly and its method of manufacture, according to the present invention, will now be described, by way of example, with reference to the accompanying drawings, in which:

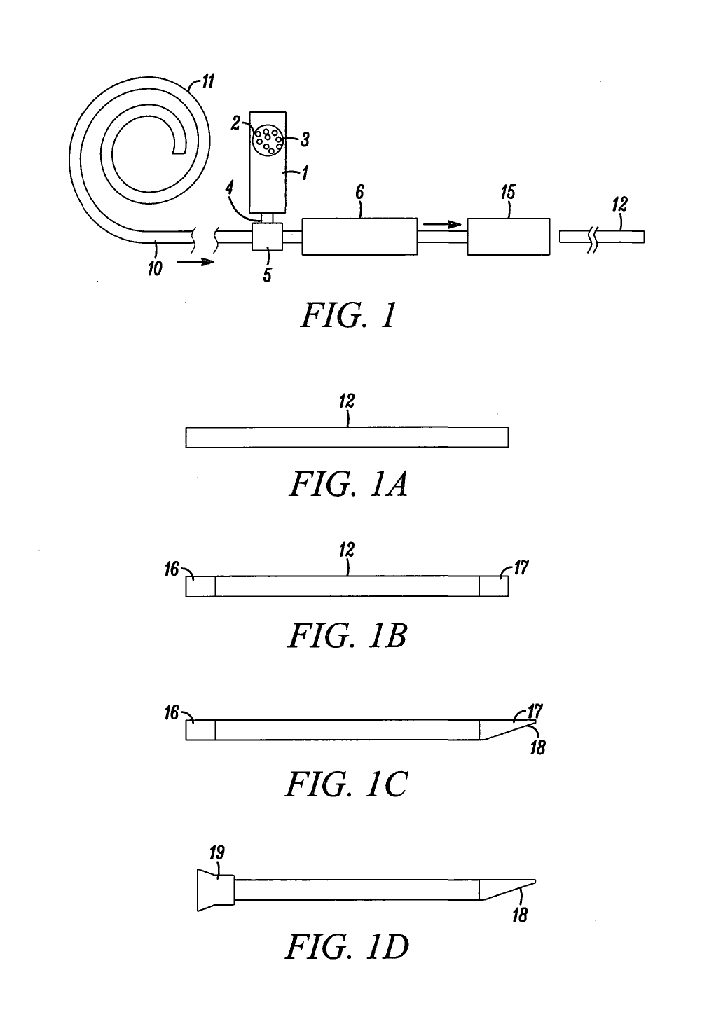

FIG. 1 illustrates a first form of apparatus for use in manufacture of the needle assemblies and illustrates various stages in the manufacture;

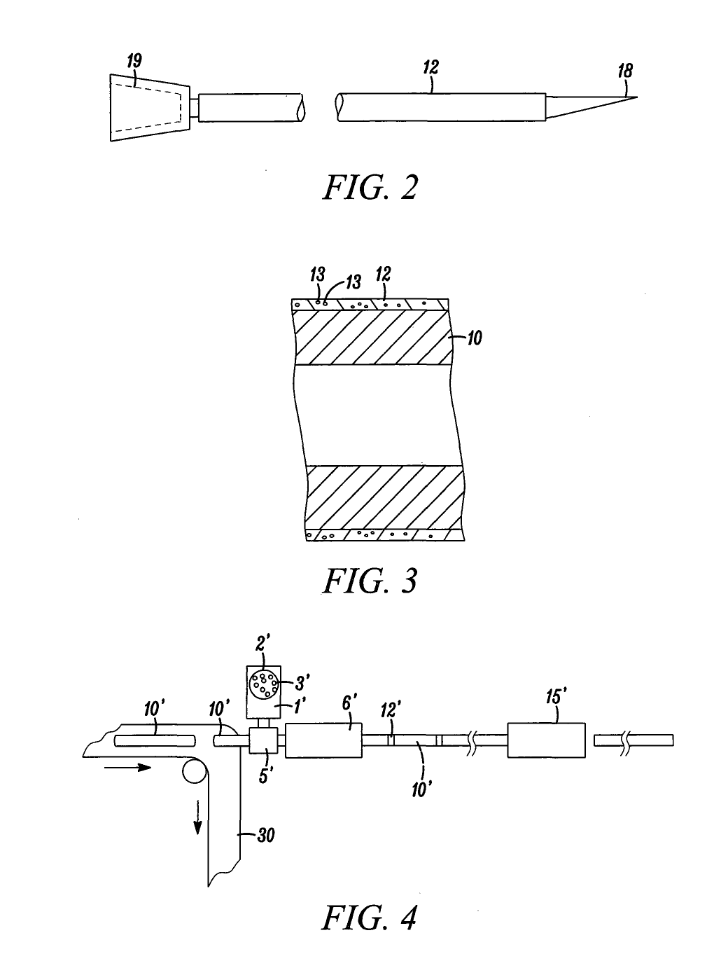

FIG. 2 is a side elevation view of a needle assembly after manufacture;

FIG. 3 is an enlarged cross-sectional side elevation view of a part of the needle assembly shown in FIG. 2; and

FIG. 4 illustrates an alternative apparatus for use in manufacturing the needle assemblies.

With reference first to FIGS. 1 to 3 there is shown extruder apparatus for use in manufacturing needle assemblies including an extruder 1 with a hopper 2 or other supply of a thermoplastics material 3 such as in pellet form. The plastics material is preferably PEBA, nylon, PVC, polyethylene, polypropylene, polyester or polyurethane and includes a suitable foaming agent. It will be appreciated that there are other ways of forming gas bubbles or interstices such as by including gas-filled polymer or glass microparticles into the plastics material. The gas within the bubbles or interstices could be of any kind and could be a vacuum. The outlet 4 of the extruder connects with a cross-head extrusion head 5, which is supplied with a hollow metal shaft or tube 10, such as of stainless steel, which typically has a diameter of between about 0.5 mm and 2.0 mm. The shaft 10, if sufficiently flexible, may be supplied from a coiled reel 11, as shown, or, if too stiff to be coiled, may be supplied in straight lengths, such as of between about 1 m and 2 m, exceeding the length of the individual needle assemblies.

The extruder 1 heats and pumps the plastics material 3 with its foaming agent to an outer die of the cross-head extrusion head 5 so that the foamed plastics material is flowed about and deposited on the outer surface of the shaft 10. The shaft 10 emerging from the head 5 is, therefore, coated with the plastics material 3, which cools and solidifies as it passes through a cooling tank 6 to form a smooth outer layer or sleeve 12 of a plastics material containing gas bubbles 13 (FIG. 3). The size and density of the bubbles 13 are selected to ensure that the layer 12 is highly echogenic. Typically the gas bubbles 13 have a size in the range 0.1 μ to 300 μ, preferably having a size in the range 1 μ to 50 μ, and most preferably having a size in the range 5 μ to 10 μ. Because the outer layer 12 is formed directly on the shaft 10 and does not need to be manually handled, the outer layer can be relatively thin, such as between 0.01 mm and 2 mm, and preferably between 0.01 mm and 0.1 mm.

A haul-off and cutter unit 15 pulls the coated shaft 10 through the extrusion head 5 and severs it into lengths equal to that of the desired needle assemblies, typically being 50-150 mm long, as shown in FIG. 1A. A short length of the plastics outer layer 12 is then removed from both ends of the cut shafts to form opposite end regions 16 and 17 where the metal is exposed, as shown in FIG. 1B. The plastics may be removed by any conventional technique, such as by cutting a ring around the layer and stripping off the end pieces. Alternatively, techniques involving grinding, milling, laser, thermal or chemical methods may be used. One exposed end 17 is then ground to form a sharp, bevelled penetrating tip 18, as shown in FIG. 1C. A hub 19 is then attached to the opposite exposed end 16, as shown in FIG. 1D to form the finished needle assembly shown in FIG. 2. It will be appreciated that the hub 19 could be attached to the shaft 10 before forming the sharp tip 18, instead of after forming the tip.

The plastics extrusion could include two or more layers as a co-extrusion. These could include an inner layer against the metal shaft acting as a bonding layer for the bubble-filled layer. The extrusion could include an outer layer without bubbles to provide a smooth layer on top of the bubble layer and, hence, a smooth outer surface to the needle assembly. Sections of the bubble layer could be removed to give distance/depth markings.

With reference now to FIG. 4 there is shown an alternative manufacturing arrangement identical with that shown in FIG. 1 except that, instead of the metal tube or shaft being supplied to the extrusion head 5′ in lengths greater than that of the formed needle assembly, the metal shaft is pre-cut into discrete lengths 10′ equal to that of the needle assemblies before supply to the extruder. These pre-cut lengths 10′ could be supplied on a web 30 from which the shaft lengths are peeled off adjacent the extrusion head 5. Alternatively, the pre-cut lengths could be supplied in a cassette or some other temporary holder. The pre-cut lengths 10′ could either be supplied preformed with a sharpened bevelled tip or these could be formed after the extrusion process in the manner described above with reference to the extruder shown in FIG. 1. The extruded product emerging from the extrusion head 5′ consists of a plastics sleeve 12′ encasing separate lengths of the metal shafts 10′ so it is just necessary to cut the sleeve between the shafts and trim the plastic from both ends to enable the hub to be attached at one end and to expose the sharp tip at the opposite end, or to enable the sharp tip to be formed at the opposite end.

1-14. (canceled)

15. A needle assembly including a hollow needle shaft, characterized in that the assembly includes a plastics sleeve extruded onto and extending along the outside of the needle shaft, and that the extruded sleeve contains a plurality of gas bubbles within the thickness of the sleeve such that the sleeve is visible under ultrasound observation and such that the ultrasound visibility of the assembly with the sleeve is greater than that of the needle alone.

16. A needle assembly according to claim 15, characterized in that the assembly includes a hub joined at one end with the needle shaft and a sharp, penetrating tip at the opposite end.

17. A needle assembly according to claim 15, characterized in that the gas bubbles in the sleeve have a size range of 0.1 μ to 300 μ.

18. A needle assembly according to claim 17, characterized in that the gas bubbles have a size range of 1 μ to 50 μ.

19. A needle assembly according to claim 18, characterized in that the gas bubbles have a size range of 5 μ to 10 μ.

20. A needle assembly according to claim 15, characterized in that the sleeve has a thickness between 0.01 mm and 2 mm.

21. A needle assembly according to claim 15, characterized in that the sleeve has a thickness between 0.01 mm and 0.1 mm.

22. A method of manufacture of a needle assembly including the steps of providing a metal shaft and extruding on the outside of the shaft a sleeve of a plastics material containing gas bubbles within its thickness, wherein the metal shaft is formed with a sharp, penetrating tip and the gas bubbles are effective to increase ultrasound visibility of the assembly.

23. A method according to claim 22, characterized in that the sharp, penetrating tip is formed before the plastics sleeve is extruded on the shaft.

24. A method according to claim 22, characterized in that the sharp, penetrating tip is formed after the plastics sleeve is extruded on the shaft.

25. A method of manufacture of needle assemblies including the steps of supplying a hollow metal shaft to extruder apparatus, extruding around the outside of the shaft in the extruder apparatus a sleeve of a plastics material containing gas bubbles, subsequently cutting the shaft into discrete lengths, and forming a sharp, penetrating tip at one end to form needle assemblies.

26. A method of manufacture of needle assemblies including the steps of supplying to extruder apparatus a plurality of pre-cut lengths of metal shafts extruding around the outside of the shafts in the extruder apparatus a sleeve of a plastics material containing gas bubbles, subsequently cutting through the extruded sleeve to form separate shafts, each shaft being formed with a sharp, penetrating tip at one end either before or after supply to the extruder machine.

27. A needle assembly made by providing a metal shaft and extruding on the outside of the shaft a sleeve of a plastics material containing gas bubbles within its thickness effective to increase ultrasound visibility of the assembly, the metal shaft being formed with a sharp penetrating tip.

28. A method according to claim 22, characterized in that the method includes the step of attaching a hub to each shaft at the end opposite the sharp, penetrating tip.

29. A method according to claim 25, characterized in that the method includes the step of attaching a hub to each shaft at the end opposite the sharp, penetrating tip.

30. A method according to claim 26, characterized in that the method includes the step of attaching a hub to each shaft at the end opposite the sharp, penetrating tip.
